(19) United States
(12) Patent Application Publication
(10) Pub. No.: US 2005/0037065 A1
(43) Pub. Date: Feb. 17, 2005

(54) NUTRITIONAL FORMULATIONS

(75) Inventors: Mitchell I. Kirschner, St. Louis, MO (US); R. Saul Levinson, Chesterfield, MO (US)

Correspondence Address: BLACKWELL SANDERS PEPER MARTIN LLP, 720 OLIVE STREET, SUITE 2400, ST. LOUIS, MO, 63101, US

(73) Assignee: DRUGTECH CORPORATION, Wilmington, DE (US)

(21) Appl. No.: 10/709,870

(22) Filed: Jun. 2, 2004

Related U.S. Application Data
(63) Continuation-in-part of application No. 10/714,156, filed on Nov. 14, 2003.
Application No. 10/714,156 is a continuation of application No. 09/972,664, filed on Oct. 9, 2001, now abandoned.
Application No. 09/972,664 is a continuation-in-part of application No. 09/320,559, filed on May 27, 1999, now abandoned.

(51) Int. Cl. A61K 33/26; A61K 31/202; A61K 9/64

(52) U.S. Cl. 424/456; 424/646; 514/560

(57) ABSTRACT

This invention relates to novel nutritional supplements comprising essential fatty acids and iron, as well as methods related thereto.

### CROSS REFERENCE TO RELATED APPLICATIONS

This application is a continuation-in-part of U.S. patent application Ser. No. 10/714,156, filed Nov. 14, 2003 and currently pending; which application is a continuation of U.S. Patent Publication No. 2002/0044961, Ser. No. 09/972,664, filed Oct. 9, 2001, now abandoned; which is a continuation in part of Ser. No. 09/320,559, filed May 27, 1999, now abandoned, all incorporated herein by reference.

### BACKGROUND OF INVENTION

This invention is directed to novel soft gelatin encapsulated nutritional supplements, particularly soft gelatin encapsulated nutritional supplements for pregnant women containing essential fatty acids and iron, as well as vitamins and minerals. The invention is further directed to methods of using said supplements to provide nutritional support to a pregnant or nursing woman and her fetus and/or nursing child. The supplements are specifically designed to reduce the unpleasant taste, regurgitation, gastroesophageal reflux, dyspepsia, and nausea associated with the administration of traditional prenatal nutritional supplements, and processes for manufacturing said supplements.

Essential fatty acids (arachadonic acid, eicosapentaenoic acid and docosahexaenoic acid) are essential for proper development of a fetus and for proper biological functioning of the mother. Stored fatty acids supplies are biochemical building blocks that support most of the body's biochemical pathways. However, it has been documented that reduction in maternal essential fatty acid status is a known phenomenon. Otto, S. J., et al., Maternal and Neonatal Essential Fatty Acid Status in Phospholipids: An International Comparative Study, European Journal of Clinical Nutrition, April 1997, Vol. 51, No. 4, 232-242. Thus, because essential fatty acids are necessary to the development of the fetus, pregnant and/or lactating women must sustain sufficient levels of various fatty acids throughout pregnancy and lactation.

Linoleic acid and linolenic acid are precursors to the essential fatty acids and are obtained through dietary intake. Arachadonic acid, eicosapentaenoic acid (EPA) and docosahexaenoic acid (DHA) are essential fatty acids required in maintaining maternal and fetal health.

Linoleic acid is an important precursor of the omega-6 family of fatty acids. The body uses linoleic acid to synthesize an important 20-carbon fatty acid, arachidonic acid, which helps maintain the structural integrity of cell membranes.

Linolenic acid is an important precursor of the omega-3 family. The body requires this fatty acid to make eicosapentaenoic acid (EPA) and docosahexaenoic acid (DHA). Many body tissues require EPA and DHA. DHA is especially important in the retina and in the cerebral cortex of the brain. Half of the DHA in a fetus's body accumulates in the brain before birth, and half after birth, an indication of the importance of fatty acids to the fetus during pregnancy and then to the young infant during lactation.

Iron supplementation during pregnancy is routine due to the fact that iron deficiency anemia is commonly encountered in pregnant and lactating women. Such anemia may be treated with dietary therapy, where possible. However the severity of the anemia or gastric state of the woman, e.g., morning sickness, etc., may make this course impracticable. Thus iron supplements, including additional vitamins, such as vitamin B-12 and folic acid, may be administered to increase the absorption of iron.

Gastrointestinal motility problems are common in women at all stages of pregnancy. Approximately 45% to 85% of women report experiencing digestive disturbances during pregnancy. Olans, et al., Gastroesophageal Reflux in Pregnancy, Gastrointest Endosc Clin N Am 4(4):699-712 (1994). Typical symptoms experienced by pregnant women include belching, heartburn, gastroesophageal reflux, dyspepsia, regurgitation, increased sensitivity to unpleasant odors and/or tastes, nausea and vomiting. The Merck Manual, 1850-1866 (16 Ed. 1992). These symptoms are thought to be brought about, in part, by the physiological changes that occur in the female body during pregnancy.

As pregnancy progresses, gastrointestinal motility decreases due to elevated progesterone levels that cause the smooth muscles associated with the digestive tract to relax. Id. The delay in gastric emptying time and relaxation of the sphincter located at the junction of the esophagus and stomach can cause a reflux of gastric fluids into the esophagus, e.g. gastroesophageal reflux. Id. The relaxation of the diaphragmatic hiatus can exacerbate this condition. Id.

The caustic nature of the refluxate and the inability to clear the refluxate from the esophagus can cause heartburn or heartburn-like symptoms. Id. In some instances, the heartburn symptoms will be accompanied by regurgitation of the gastric contents into the mouth. The Merck Manual, 1850-1866 (16^{th} Ed. 1992).

The condition of gastroesophageal reflux may be self-perpetuating if not managed and/or treated. Because of the caustic properties of the gastric contents, repeated esophageal exposure to these substances can lead to a permanent incompetence of the esophageal sphincter. Id. Furthermore, in more serious cases, esophagitis, peptic esophageal stricture, esophageal ulcer, and Battert's metaplasia can result in a case of complicated gastroesophageal reflux. Id. Therefore, management and therapy of the condition are of the utmost priority.

The gastrointestinal disturbances associated with pregnancy are normally mild in degree and viewed as a natural part of the pregnancy experience. However, these facts do not lessen the discomfort experienced by pregnant women or the seriousness of the potential complications of the condition. Furthermore, as with any course of medical treatment in pregnant women, a primary concern is the potential teratogenicity of the proposed drug therapy. Many gastrointestinal medications are either known teratogens or have not been adequately studied with regards to their effect upon pregnant humans.

It has been noted that medications used in the treatment of gastroesophageal reflux are not routinely or vigorously tested in randomized, controlled trials in pregnant women because of ethical and medico-legal concerns. Broussard, et al. Treating Gastroesophageal Reflux Disease during Pregnancy and Lactation: What are the Safest Therapy Options, Drug Saf, 19(4): 325-37 (1998). For example, the cholinergic antagonist Cystospaz®, available from PolyMedica Pharmaceuticals (U.S.A.), Inc., which is of the class of drugs which can be prescribed for gastroesophageal reflux due to their positive effect upon esophageal sphincter pressure, is not recommended for use in pregnant women, because animal reproductive studies have not been conducted. Furthermore, it is not known whether CYSTOSPAZ® Tablets or CYSTOSPAZ-M®Capsules, can cause fetal harm when administered to a pregnant woman. Physicians' Desk Reference, 2526-7 (53d Ed. 1999).

Other cholinergic antagonists are provided with similar precautions. Donnatal®, available from A.H. Robins Company, is not recommended for administration to pregnant women due to the lack of adequate animal reproduction studies, and also because the effect of the drug on the fetus is not known. Id. at 2636. Kutrase®, available from Schwarz Pharma, Inc., Levsin®, also available from Schwarz Pharma, Inc. and Robaxisal®, available from A.H. Robins Company, all carry similar precautions regarding prescription to pregnant and/or lactating women. Id at 2907; See also, Id. at 2910; See also, Id. at 2646.

As a result, most physicians initially begin managing gastrointestinal disturbances in pregnant women with aggressive lifestyle modification and dietary changes rather than drug therapy. Katz, et al., Gastroesophageal Reflux Disease during Pregnancy, Gastroenterol. Clin. North. Am., 27(1): 153-67 (1998). While this course of therapy is primarily due to the concern of exposing the fetus to teratogenic substances via drug therapy, it has been discovered that lifestyle and dietary management are often extremely effective in precipitating relief. Katz, et al. Gastroesophageal Reflux Disease during Pregnancy, Gastroenterol. Clin. North. Am. 27(1):153-67 (1998).

Dietary management consists of isolating those foods or classes of foods that bring about the symptoms of gastroesophageal reflux. The Merck Manual, 749 (16^{th} Ed. 1992). Typically, the common foods which aggravate the condition are fried or fatty foods, caffeinated beverages or foods, for example coffee and chocolate, and spicy foods. It is thought that these foods stimulate acid production and/or reduce lower esophageal sphincter competence. Id.; see also, Nebel, et al., Symptomatic Gastroesophageal Reflux: Incidence and Precipitating Factors, Am. J. Dig. Dis. 21(11): 953-6 (1976).

Furthermore, it has been discovered that gastrointestinal relief can be brought about by directing the pregnant woman to eat small portions at frequent intervals and to increase the amount of carbohydrates while simultaneously decreasing her fat intake. Morton, Treating Nausea and Vomiting in Pregnancy, Am. Fam. Physician, 48(7): 1279-84 (1993). Other general recommendations include instituting a bland diet, avoiding bothersome food odors and omitting prenatal vitamins from the dietary regimen. Id.

The omission of prenatal vitamins is a problematic recommendation for the pregnant woman. While it is acknowledged that vitamin supplements can cause uncomfortable gastrointestinal effects, i.e., gagging, regurgitation, gastroesophageal reflux, dyspepsia, and/or nausea, and can be unpleasant to take due to taste, smell, size and/or the texture of the tablet, it is also a well established fact that pregnant women have heightened nutritional requirements. A mother's body provides the environment in which development of the embryo and fetus occur. See Understanding Nutrition, 479-480 (Whitney and Rolfes Eds. 6^{th} Ed., 1993). Accordingly, the mother's nutritional status during pregnancy directly impacts the development of the fetus and embryo and is therefore implicated with regard to the occurrence of birth defects. See Id.

In particular, during the first 20-25 days of pregnancy, the placenta is not yet formed and fetal circulation is not yet established. Therefore, during this period the fetus is nourished via digested maternal uterine cells and the diffusion of blood exudates. See Schorah, Importance of Adequate Folate Nutrition in Embryonic and Early Fetal Development, Vitamins and Minerals in Pregnancy and Lactation, 167-176 (Berger, Ed., Vol. 16, 1988). It is believed that a good nutrient supply during the first 20-25 days of pregnancy is necessary to provide optimal concentrations of essential micronutrients to the endometrium. See Id.

Furthermore, increased occurrences of birth defects have been linked to inadequate maternal nutrition. Cases of infants born with a neural tube defect, i.e., spina bifida or anacephaly, have been documented in women with various nutritional deficiencies, primarily low blood folic acid and vitamin C concentrations. Smithells, Vitamin Deficiencies and Neural Tube Defects, Arch. Dis. Child, 51:944-50 (1976).

The importance of the nutritional status of pregnant women is evident in the number of prenatal vitamins currently available. The Physicians' Desk Reference describes various vitamin and mineral supplements for use by pregnant women. For example, Nestabs® CBF prenatal formula, available from The Fielding Company, contains 4,000 I.U. of vitamin A, 400 I.U. of vitamin D, 30 I.U. of vitamin E, 120 mg Of vitamin C, 1 mg of folic acid, 3 mg of thiamine, 3 mg of riboflavin, 20 mg of niacinamide, 3 mg of pyridoxine, 8 mcg of vitamin B₁₂, 20 mg of calcium, 100 mcg of iodine, 15 mg of zinc, and 50 mg of iron per dose. NESTABS® CBF are expressly formulated for use during pregnancy and lactation and are available only in tablet form. See Physicians' Desk Reference, 1011 (53d Ed., 1999).

Matema®, prenatal vitamin and mineral formula, available from Lederle Laboratories, contains 5,000 I.U. of vitamin A, 400 I.U. of vitamin D, 30 I.U. of vitamin E, 120 mg of vitamin C, 1 mg of folic acid, 3 mg of vitamin B₁, 3.4 mg of vitamin B₂, 10 mg of vitamin B₆, 20 mg of niacinamide, 12 mcg of vitamin B₁₂, 30 mcg of biotin, 10 mg of pantothenic acid, 200 mg of calcium, 150 mcg of iodine, 27 mg of iron, 25 mg of magnesium, 2 mg of copper, 25 mg of zinc, 25 mg of chromium, 25 mg of molybdenum, 5 mg of manganese, and 20 mcg of selenium per dose. Matema® is designed to provide vitamin and mineral supplementation prior to conception, throughout pregnancy and during the postnatal period for both lactating and nonlactating mothers and is available in tablet form only. See Id. at 1522-3.

Enfamil® Natalins® RX multivitamin and multimineral supplement, available from Mead Johnson Nutritionals, Mead Johnson & Company, provides 4000 I .U. of vitamin A, 80 mg of vitamin C, 400 I.U. of vitamin D, 15 I.U. of vitamin E, 1.5 mg of thiamin, 1.6 mg of riboflavin, 17 mg niacin, 4 mg of vitamin B₆, 1 mg of folic acid, 2.5 mcg of vitamin B₁₂, 30 mcg of biotin, 7 mg of pantothentic acid, 200 mg of calcium, 54 mg of iron, 25 mg of zinc, and 3 mg of copper per dose. Enfamil® Natalins® RX are formulated Ato supplement the diet during pregnancy of lactation and are available only in tablet form. See Id. at 1692.

Prenate® Ultra prenatal vitamins, available from Sanofi Pharmaceuticals, Inc., contain 90 mg of elemental iron, 150 mcg of iodine, 200 mg of calcium, 2 mg of copper, 25 mg of zinc, 1 mg of folic acid, 2700 I.U. of vitamin A, 400 I.U. of vitamin D₃, 30 I.U. of vitamin E, 120 mg of vitamin C, 3 mg of vitamin B₁, 304 mg of vitamin B₂, 20 mg of vitamin B₆, 12 mcg of vitamin B₁₂, 20 mg of niacinamide, and 50 mg of docusate sodium per dose. Prenate® Ultra is indicated for use in improving the nutritional status of women throughout pregnancy and in the postnatal period for both lactating and nonlactating mothers and is only available in tablet form. See Id. at 2802.

Niferex®-PN formula, available from Schwarz Pharmaca, Inc., contains 60 mg of iron, 1 mg of folic acid, 50 mg of vitamin C, 3 mcg of vitamin B₁₂, 4,000 I.U. of vitamin A, 400 I.U. of vitamin D, 2.43 mg of vitamin B₁, 3 mg of vitamin B₂, 1.64 mg of vitamin B₆, 10 mg of niacinamide, 125 mg of calcium, and 18 mg of zinc per dose. Niferex®-PN is indicated for prevention and/or treatment of dietary vitamin and mineral deficiencies associated with pregnancy and lactation and is only available in tablet form. See Physicians' Desk Reference, (53d Ed., 1999) 2916-7.

Niferex®-PN Forte formula, also available from Schwarz Pharmaca, Inc., contains 60 mg of iron, 1 mg of folic acid, 50 mg of vitamin C, 3 mcg of vitamin B₁₂, 5,000 I.U. of vitamin A, 400 I.U. of vitamin D, 30 I.U. of vitamin E, 80 mg of vitamin C, 1 mg of folic acid, 3 mg of vitamin B₁, 3.4 mg of vitamin B₂, 4 mg of vitamin B₆, 20 mg of niacinamide, 12 mcg of vitamin B₁₂, 250 mg of calcium, 200 mcg of iodine, 10 mg of magnesium, 2 mg of copper, and 25 mg of zinc per dose. Niferex®-PN is indicated for prevention and/or treatment of dietary vitamin and mineral deficiencies associated with pregnancy and lactation and is only available in tablet form. See Id. at 2917-8.

Advanced Formula Zenate® prenatal multivitamin/mineral supplement, available from Solvay Pharmaceuticals, Inc., contains 3,000 I.U. of vitamin A, 400 I.U. of vitamin D, 10 I.U. of vitamin E, 70 mg of vitamin C, 1 mg of folic acid, 1.5 mg of vitamin B₁, 1.6 mg of vitamin B₂, 17 mg of niacin, 2.2 mg of vitamin B₆, 2.2 of vitamin B₁₂, 200 mg of calcium, 175 mcg of iodine, 65 mg of iron, 100 mg of magnesium, and 15 mg of zinc per dose. Advanced Formula Zenate® is a dietary adjunct in nutritional stress associated with periconception, pregnancy and lactation and is only available in tablet form. See Id. at 3128.

Precare® prenatal multi-vitamin/mineral formula, available from Ther-Rx Corporation, contains 50 mg of vitamin C, 250 mg of calcium, 40 mg of iron, 6 mcg of vitamin D, 3.5 mg of vitamin E, 2 mg of vitamin B₆, 1 mg of folic acid, 50 mg of magnesium, 15 mg of zinc and 2 mg of copper per dose. Precare® is indicated to provide vitamin and mineral supplementation throughout pregnancy and during the postnatal period-for both lactating and nonlactating mothers and is available only in caplet form. See Id. at 3163.

Natafort® prenatal multivitamin, available from Warner Chilcott Laboratories, contains 1,000 I.U. of vitamin A, 400 I.U. of vitamin D₃, 11 I.U. of vitamin E, 120 mg of vitamin C, 1 mg of folic acid, 2 mg of thiamine mononitrate, 3 mg of riboflavin, 20 mg of niacinamide, 10 mg of vitamin B₆, 12 mcg of vitamin B₁₂, and 60 mg of iron per dose. Natafort® is designed to provide vitamin and mineral supplementation throughout pregnancy and during the postnatal period, for both the lactating and non-lactating mother and is only available in tablet form. See Id. at 3212.

PrimaCare, a nutritional supplement available from KV Pharmaceuticals, the assignee of the present invention, comprises essential fatty acids, vitamins and minerals and requires two dosage forms, a soft gelatin capsule and a tablet.

Soft gelatin capsule dosage forms are flexible, one-piece, hermetically sealed soft shells, comprised of gelatin, a plasticizer, and a small quantity of water and which contains a fill, of one or more active ingredients in combination to form a liquid, suspension or a semi-solid center. Soft gelatin technology has been previously described in various references. For example, Yu et al., U.S. Pat. No. 5,071,643, disclose a solvent system for enhancing the solubility of acidic, basic, or amphoteric pharmaceutical agents to produce a highly concentrated solution suitable for soft gelatin filling or two piece encapsulation. The solvent system comprises polyethylene glycol containing 0.2-1.0 mole equivalent pharmaceutical agent and 1-20% water. Glycerin or polyvinylpyrrolidone may be added to further enhance the solubility of certain drugs. The solvent system is capable of enhancing the solubility of pharmaceutical agents 40-400%.

Stone, U.S. Pat. No. 5,827,535, discloses a soft gelatin bearing an impressed graphic representation, such as a letter, name, logo, pictorial representation and the like and a method for making such a soft gelatin.

Ratko et al., U.S. Pat. Nos. 5,422,160 and 5,246,635, disclose a soft gelatin having a texture on at least a portion of its surface and a process and apparatus for the manufacture of such a soft gelatin.

Steele et al., U.S. Pat. No. 5,200,191, disclose a soft gelatin manufacturing process comprising subjecting encapsulated soft gelatins to a stress relieving step, wherein the soft gelatins are placed in a drying tunnel and exposed to heightened temperature and humidity conditions.

Coapman et al., U.S. Pat. No. 5,141,961, disclose a process for solubilizing difficultly soluble pharmaceutical actives in a mixture of polyethylene glycol and polyvinylpyrolidone in the absence of external heat or water.

Cimiluca, U.S. Pat. No. 5,641,512, discloses a soft gelatin capsule composition comprising an analgesic in a soft shell containing a xanthine derivative, such as caffeine.

Yu et al., U.S. Pat. No. 5,360,615, disclose a solvent system for enhancing the solubility of acidic, basic, or amphoteric pharmaceutical agent to produce a highly concentrated solution suitable for soft gelatin filling or two piece encapsulation. The solvent system comprises polyethylene glycol containing 0.2-1.0 mole equivalents of an ionizing agent per mole equivalent pharmaceutical agent and 1-20% water.

The compositions and methods discussed above are deficient in various aspects. Primarily, the compositions are not specifically formulated for administration of fatty acids and iron in soft gelatin dosage form. Even the above discussed references, which recognize the need for an easier to swallow form of prenatal vitamin, are limited to coated tablet or caplet forms and are not optimal for minimizing unpleasant taste and/or smell, regurgitation, gastroesophageal reflux, dyspepsia, and/or nausea and maximizing ease of swallowing or ingestion. Furthermore, the soft gelatin formulations which are discussed do not offer any guidance with regard to formulating specific nutritional compositions containing fatty acids and iron, as well as other vitamins and minerals for the prenatal patient. Thus, these references are inadequate with regard to improving oral vitamin and mineral supplement administration for pregnant women. Further, the presence of iron in a soft gelatin capsule tends to crosslink the gelatin rendering it insoluble in water. This results in failure to dissolve and release its contents after ingestion. Finally, previously disclosed compositions do not provide guidance with regard to optimal means of achieving a biologically-active soft gelatin dosage form of prenatal vitamin.

Therefore, there remains a need in the art for a soft gelatin prenatal vitamin and mineral supplement which delivers fatty acids and iron, along with vitamins and other minerals, which has a minimal negative effect upon the gastrointestinal tract of the patent, as well as supports the general health of the patient. Moreover, there is a particular need for soft gelatin formulations that promote the good health of the expectant mother and are pleasant to ingest, and thus will provide a higher degree of patient compliance while simultaneously minimizing the cost to the patient.

It is also particularly desirable to have available formulations for addressing the nutritional needs of pregnant women which are designed to have a minimized impact upon the gastrointestinal system, specifically by providing a formulation which delivers fatty acids and iron over an extended period of time. Because of the sensitive nature of this system during pregnancy and the desire to reduce or avoid medication during pregnancy, such soft gelatin formulations are advantageous in that they do not provoke gastrointestinal disturbances. Thus, there is a general overall need for a fundamentally new, safe and effective approach to addressing the physiological needs of pregnant women required to or desirous of partaking in a prenatal vitamin and mineral regimen but are unable to do so because of gastrointestinal system sensitivity.

### SUMMARY OF INVENTION

In an aspect of the present invention a soft gel nutritional supplement for administration to a pregnant or lactating woman is provided. The nutritional supplement comprises at least one essential fatty acid selected from the group consisting of essential fatty acids, precursors thereof, derivatives thereof and mixtures thereof; and at least one pharmaceutically acceptable iron compound, wherein said nutritional supplement is provided in a soft gelatin shell dosage.

In another aspect of the invention a method of making a soft gel nutritional supplement for administration to said pregnant or lactating woman is provided. The method comprises at least one essential fatty acid selected from the group consisting of essential fatty acids, precursors thereof, derivatives thereof and mixtures thereof; and at least one pharmaceutically acceptable iron compound, wherein said nutritional supplement is provided in a soft gelatin shell dosage.

In yet another aspect of the present invention a method for administering a nutritional supplement for administration to a pregnant or lactating woman is provided. The method comprises orally administering a soft gel capsule comprising at least one essential fatty acid selected from the group consisting of at least one essential fatty acid, at least one essential fatty acid precursor, at least one derivative of an essential fatty acid and mixtures thereof; and at least one pharmaceutically acceptable iron compound, wherein said nutritional supplement is provided in a soft gelatin shell dosage.

These are merely illustrative aspects of the present invention.

### DETAILED DESCRIPTION

As used herein, soft gelatin may refer to a one-piece, hermetically sealed soft or semi-soft gelatin shell containing a fill, in particular a liquid, a suspension or a semi-solid.

Unpleasant taste may refer to the bothersome taste normally associated with oral dosage forms containing nutritional compounds or any taste which is typically thought of as not palatably desirable to most people, but in particular pregnant or nursing women.

Difficulty in swallowing or ingestion may refer to the hindered ability to orally consume nutritional compounds. Primarily this may be due to the supplement's unpleasant taste and/or smell, gastrointestinal sensitivity or some other incompatibility between the patient's physiology and the physical properties of the nutritional compounds, without limitation.

Biologically-active core composition may refer to a liquid, suspension or semi-solid composition which is contained within the soft gelatin coating and is comprised of nutritional compound suspended in an edible oil or polymer and which further may be used for treatment, prevention, diagnosis, cure or mitigation of disease or illness, to effect anatomical structure or physiological function, or alter the impact of external influences upon the body.

Nutritional compound may refer to any compound which provides nourishment to cells of the body and developing embryo or fetus, as well as a nursing child, including without limitation: any vitamin, mineral, enzyme, trace element, micronutrient, fatty acid, triglyceride, amino acid, herbal compounds, electrolyte, protein, carbohydrate, derivative thereof or combinations thereof.

The present inventive subject matter is based, in part, upon the discovery that pregnant women have specific nutritional requirements and that there are substantial physiological benefits attained by fulfilling these requirements. Particularly, the invention is concerned with the administration of essential fatty acids and forms of iron to pregnant and/or nursing women.

Of additional interest to the inventive subject matter is the discovery that the ability to meet the nutritional requirements of pregnant women is sometimes hindered due to the increased sensitivity of the pregnant woman's gastrointestinal tract. However, minimizing this sensitivity is possible through implementation of lifestyle and dietary modifications. The products of the inventive subject matter provide optimum nutritional components and are provided in a dosage form that takes into account the increased gastrointestinal sensitivity of pregnant women.

Without being limited by theory, the compositions and methods of the present inventive subject matter may be effective because they provide a source of essential fatty acids and iron which are critical to the maintenance of maternal health and development of the child. Also, said nutrients are provided in a dosage form which is designed to have a low impact upon the gastrointestinal tract, in that the dosages are of soft and flexible design and minimize unpleasant taste and/or smell. Alternatively, the compositions and methods may be effective because they do not initiate, stimulate or act as catalysts to reactions having a negative effect upon the gastrointestinal tract.

The nutritional supplements of the present inventive subject matter contain specific nutritional compositions for administration to pregnant women to alleviate nutritional deficiencies likely to occur during pregnancy. Further, the present inventive subject matter also satisfies specific vitamin and mineral requirements, the absence of which have been found to cause birth defects, as well as provide for general health during pregnancy. The formulations of the inventive subject matter optimize the nutritional benefits of supplementation as required by the physiological stresses of pregnancy.

The nutritional compositions of the present inventive subject matter are provided in a dosage form, i.e., soft gelatin, for administration to pregnant women which minimizes unpleasant taste, regurgitation, gastroesophageal reflux, dyspepsia, nausea, or difficulty in swallowing or ingesting nutritional agents during pregnancy. The effectiveness of the soft gelatin dosage form in relation to its low impact effect upon the gastrointestinal tract appears to be related to the dosage's small size and flexible, soft physical properties. The soft gelatins of the present inventive subject matter have a smooth outer surface, which has elastic properties that provide for minimal resistance in swallowing. As such, the soft gelatins have a lesser potential to negatively impact the esophageal sphincter and thereby cause or exacerbate the condition of gastroesophageal reflux. These same properties, as well as the pre-dispersion of the nutritional compositions in the core matrix, reduce the reactivity of the actives to the acidic gastrointestinal environment, and thus lend to reduced incidences of reflux and regurgitation phenomena. Furthermore, the gelatin coating of the soft gelatins minimizes the unpleasant taste and/or smell commonly associated with traditional vitamin and mineral supplements and thereby reduces regurgitation, dyspepsia, nausea and gagging associated with these negative traits.

The nutritional compositions of the present inventive subject matter are formulated to provide for optimal health during pregnancy and to minimize any potential negative impact upon the gastrointestinal tract. The extent to which this negative impact is reduced by use of the soft gelatin formulas is mitigated by numerous external factors, such as the following non-limiting examples: stress, alcohol intake, caffeine intake, smoking, poor diet management, poor patient compliance, and the like. Moreover, the effectiveness of the compositions may vary from individual to individual for a wide array of reasons, such as genetic predisposition, health factors, and the like, without limitation.

is difficult to quantify the minimizing effect upon unpleasant taste, regurgitation, gastroesophageal reflux, dyspepsia, nausea, or difficulty swallowing or ingesting of the soft gelatin nutritional agents. However, the average healthy pregnant woman suffering from the normal gastrointestinal disturbances associated with pregnancy, i.e., uncomplicated incidences of heartburn, gastroesophageal reflux, dyspepsia, nausea, regurgitation, gagging, and the like, without limitation, may be able to minimize these symptoms through use of the present formulations. Furthermore, even for pregnant women who are experiencing gastrointestinal disturbances to a more pronounced than what would be classified as normal may find the formulations of the present inventive subject matter have a positive effect upon these symptoms, particularly where the gastrointestinal distress is caused or exacerbated by the ingestion of traditional vitamin and mineral tablets or where their condition has made it impossible to ingest traditional tablet form prenatal supplements.

The present inventive subject matter contemplates the inclusion of a viscous biologically-active core composition that is comprised of a nutritional compound uniformly suspended in an edible oil or a polymer. Preferably, the nutritional compound is about 2 percent to 98 percent by weight of the biologically-active core composition. More preferably, the nutritional compound is about 3 percent to 97 percent by weight of the biologically-active core. Most preferably, however, the nutritional compound is about 4 percent to 96 percent by weight of the biologically-active core.

In alternative embodiments of the present invention the dosage form may take the form of other dosage forms as are well known in the art.

The compositions of the present inventive subject matter include essential fatty acids. Essential fatty acids are any biologically useful fatty acid, and may include polyunsaturated short, medium or long chain fatty acids, omega-3, omega-6, and omega-9 fatty acids as well as precursors and derivatives of any fatty acid, such as omega-3, omega-6, and omega-9 fatty acids. Such fatty acids and precursors include arachidonic acid, eicosapentanoic acid, docosahexanoic acid, oleic acid, linolenic acid, and linoleic acid. Fatty acids of the present invention may be from any source, including, without limitation, natural or synthetic oils, fats, waxes or combinations thereof. Moreover, the fatty acids herein may be derived, without limitation, form nonhydrogenated oils, partially hydrogenated oils, fully hydrogenated oils, or combinations thereof. Nonlimiting exemplary sources of fatty acids include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba oil, tallow, beef tallow, butter, chicken fat, lard, dairy butter fat, shea butter, or combinations thereof. Specific non-limiting exemplary fish or marine oils include shell fish oil, tuna oil, mackerel oil, salmon oil, menhaden oil, anchovy oil, herring oil, trout oil, sardine oil, oils derived from seaweed or kelp, or combinations thereof.

In one embodiment of the present invention the oils are the product of algae. The use of macroalgae, primarily occurring in the sea, from the families of brown, red and green algae is utilized as a source of EFA's in U.S. Pat. No. 5,539,133, incorporated herein by reference. Of these, those from the Phaeoophyceae and Rhodophyceae families are of special interest. However, certain species are also used for human nutrition in other parts of the world, above all in the coastal countries of Northern Europe and East Asia (Japan). These macroalgae can be found in many continental shelf zones of the ocean and are available in practically unlimited quantities. A few macroalgae species are also intentionally cultivated in partitioned-off areas of the sea (aquaculture).

It has now surprisingly been found that lipids with a high proportion of LCPs can be extracted from these macroalgae in an economical way, if an organic solvent or a condensed gas is used. Moreover, the macroalgae are comminuted, in particular ground, before the actual extraction, so that the raw material obtained from these macroalgae and used in the method of the invention has a particle size of 50 mm. Furthermore, the macroalgae are dried either before or after the comminution, so that their water content amounts to 50 weight %.

Omega-3 and omega-6 fatty acid precursors are biochemical substances that precede and are forerunners to the more stable and definitive products, i.e., omega-3 and omega-6 fatty acids. These biochemical substances include, without limitation, linolenic and linoleic acids.

The fatty acid status of a pregnant and/or nursing mother is significant for development of the fetal brain, immunological system and cardiovascular system, and have some role to play in every organ of the body of the fetus or nursing infant. Linoleic acid is the most important member of the omega-6 family of fatty acids. The body uses linoleic acid to synthesize an important 20-carbon fatty acid, arachidonic acid, which helps maintain the structural integrity of cell membranes. Further, fatty acids also serve as signals inside the cell independently of cell membranes. Absolute and relative levels of essential fatty acids determine their biological effects. Thus, it is critical that proper levels be maintained by pregnant women.

The present inventive subject matter also includes an iron providing material or material. These may be selected from carbonyl iron, soluble iron salts, slightly soluble iron salts, insoluble iron salts, chelated iron, and iron complexes. Preferred chelated iron complexes are the subject of U.S. Pat. Nos. 4,599,152 and 4,830,716. In an alternative embodiment, iron that does not react with the essential fatty acids of the present invention or the gelatin comprising the soft shell is utilized. Illustrative examples of non-reactive iron include carbonyl iron, as well as iron compounds that have been encapsulated by methods well known in the art to prevent reaction with the essential fatty acids. In a preferred, non-limiting aspect of the present inventive subject matter, the soluble iron salts that may be encapsulated are selected from the group consisting of ferric hypophosphite, ferric albuminate, ferric chloride, ferric citrate, ferric oxide saccharate, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

In another preferred, non4imiting aspect of the present inventive subject matter, the slightly soluble iron salts are selected from the group consisting of ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycermphosphate, ferric saccharate, ferric hydroxide saccharate, ferric manganese saccharate, ferrous ammonium sulfate, other pharmaceutically acceptable iron salts, and combinations thereof. As discussed above, these iron salts may be encapsulated if a non-reactive iron is desired.

In yet another preferred, non-limiting aspect of the present inventive subject matter, the insoluble iron salts are selected from the group consisting of ferric sodium pyrophosphate, ferrous carbonate, ferric hydroxide, ferrous oxide, ferric oxyhydroxide, ferrous oxalate, other pharmaceutically acceptable iron salts and combinations thereof. As discussed above, these iron salts may be encapsulated if a non-reactive iron is desired.

In still yet another preferred, nonlimiting aspect of the present inventive subject matter, the iron complexes are selected from the group consisting of polysaccharide-iron complex, methylidine-iron complex, EDTA-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, MGD-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-PDTA ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof. A preferred iron is disclosed in U.S. Pat. No. 4,599,152 and U.S. Pat. No. 4,830,716 which are incorporated herein by reference in their entirety.

The formulations of the present inventive subject matter may contain vitamin B₆ or derivatives thereof. Derivatives of vitamin B₆ include compounds formed from vitamin B₆ which are structurally distinct from vitamin B₆, but which retain the active function of vitamin B₆. Such derivatives include, without limitation, pyridoxine, salts of vitamin B₆, alkaline salts of vitamin B₆, chelates of vitamin B₆, combinations thereof and the like. The vitamin B₆ may be present in a single form or in various different forms in combination within the present compositions. The specific amount of vitamin B₆ in the compositions is adjusted based on the type of dosage form utilized, i.e., immediate release or controlled release. In an illustrative embodiment the B6 comprises about 10 mg to about 150 mg.

In the case of the immediate release compositions, the amounts of vitamin B₆ in the compositions preferably range from about 1 mg to about 115 mg. More preferably, the amounts of vitamin B₆ in the immediate release compositions range from about 2 mg to about 110 mg. Even more preferably, the amounts of vitamin B₆ in the immediate release compositions range from about 3 mg to about 107 mg. Most preferably, the amounts of vitamin B₆ in the immediate release compositions range from about 4 mg to about 105 mg.

The amount of vitamin B₆ present in the controlled release compositions of the present inventive subject matter, preferably range from about 75 mg to about 125 mg. More preferably, the amount of vitamin B₆ in the controlled release compositions is about 85 mg to about 115 mg. Even more preferably, the amount of vitamin B₆ in the controlled release compositions is about 90 mg to about 110 mg. Most preferably, the amount of vitamin B₆ in the controlled release compositions is about 95 mg to about 105 mg.

The compositions of the present inventive subject matter may include a folic acid compound or derivative thereof. The derivatives of folic acid include folacin, pteroylglutamic acid, as well as compounds formed from folic acid which are structurally distinct from folic acid, but which retain the active function of folic acid. Non-limiting examples of such derivatives include: salts of folic acid, chelates of folic acid, combinations thereof and the like. The folic acid may be present in a single form or in various different forms in combination within the present compositions. Folic acid in the present compositions may be presented in various types of dosage forms, for example and without limitation, immediate release or controlled release. Extended release folic acid may be included in the present compositions, because such folic acid minimizes gastrointestinal side effects. The amounts of folic acid preferably range from about 0.4 mg to about 5.0 mg. More preferably, the amount of folic acid in these compositions is about 0.5 mg to about 4 mg. Most preferably, the amount of folic acid in these compositions is about 1 mg to about 3 mg. Hereinafter, the use of the terms folic acid and folate are deemed to include precursors, derivatives and metabolites thereof.

The folic acid or folate of the present invention may include a composition that includes one or more natural isomers of reduced folate. The natural isomers of reduced folate may be selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl(6R)-tetrahydrofolic acid, 5-formimino-(6S)-tetrahydrofolic acid, and polyglutamyl derivatives thereof, which are the subject of U.S. Pat. Nos. 5,997,915 and 6,254,904. All patents and applications cited herein are incorporated by reference.

The compositions of the present inventive subject matter may include a calcium compound or derivative thereof. The addition of calcium is beneficial nutritionally, and the calcium compound minimizes stomach upset, as well as increases the bioavailability of folic acid when present in the composition. The derivatives of calcium include, without limitation, calcium carbonate, calcium sulfate, calcium oxide, calcium hydroxide, calcium apatite, calcium citrate-malate, calcium gluconate, calcium lactate, calcium phosphate, dicalcium phosphate, tricalcium phosphate, calcium levulinate, bone meal, oyster shell, as well as compounds formed from calcium which are structurally distinct from calcium, but which retain the active function of calcium. Non-limiting examples of such derivatives include: salts of calcium, chelates of calcium, combinations thereof and the like. The calcium may be present in a single form or in various different forms in combination within the present compositions. Preferably, the supplement will contain about 50.0 mg to about 1,000 mg of calcium. More preferably, the supplement will contain about 75 mg to about 500 mg of calcium.

The compositions of the present invention achieve maintenance of essential fatty acid status in pregnant and/or nursing women through one or more natural biological pathways. For example, the arachidonic acid cascade may play a significant role in the enrichment of the breast milk. Specifically, in the arachidonic acid cascade, linoleic acid is converted first to gamma-linolenic acid and then to further metabolites such as dihomo-gamma-linolenic acid and arachidonic acid which are precursors of 1 and 2 series prostaglandin respectively.

The first fatty acid compound is selected from the group consisting of a linoleic acid compound, a In an illustrative, non-limiting embodiment the present composition contains at least two fatty acid compounds linolenic acid compound, derivatives thereof and combinations thereof. In one embodiment the first fatty acid compound preferably comprises about 10 mg to about 1000 mg, with about 50 mg to about 500 mg being more preferred and about 100 mg to about 300 mg being most preferred.

The second fatty acid compound is selected from the group consisting of a eicosapentanoic acid, docosahexaenoic acid compound, an omega-3 fatty acid compound, an omega-2 fatty acid compound, derivatives thereof and combinations thereof. It is preferred that when the first fatty acid compound is linoleic acid or a derivative thereof and the second fatty acid compound is an omega-6 fatty acid. In one embodiment the second fatty acid compound preferably comprises about 10 mg to about 1000 mg, with about 50 mg to about 500 mg being more preferred and about 100 mg to a about 300 mg being most preferred.

Preferably, the weight ratio of the first fatty acid to the second fatty acid is about 1:0.001 to 50. More preferably, the weight ratio of the first fatty acid compound to the second fatty acid compound is about 1:0.1 to 10. Even more preferably, the weight ratio of the first fatty acid compound to the second fatty acid compound is about 1:0.9 to 2.5. Most preferably, the weight ratio of the first fatty acid compound to the second fatty acid compound is about 1:1 to 2.

The fatty acids of the present inventive subject matter may be used as such or as biologically acceptable and physiologically equivalent derivatives as, for example, detailed later herein. Reference to any of the fatty acids including reference in the claims is to be taken as including reference to the acids when in the form of such derivatives. Equivalence is demonstrated by entry into the biosynthetic pathways of the body as evidenced by effects corresponding to those of the acids themselves or their natural glyceride esters. Thus, indirect identification of useful derivatives is by their having the valuable effect in the body of the fatty acid itself, but conversion, for example, of gamma-linolenic acid to dihomo-gamma-linolenic acid and on to arachidonic acid can be shown directly by gas chromatographic analysis of concentrations in blood, body fat, or other tissue by standard techniques, well known to persons of ordinary skill in the art to which the present inventive subject matter pertains.

Derivatives of linoleic acid, as used in the present inventive subject matter, include, without limitation, salts of linoleic acid, alkaline salts of linoleic acid, esters of linoleic acid, and combinations thereof. Derivatives of linolenic acid, as used in the present inventive subject matter, include, without limitation, salts of linolenic acid, alkaline salts of linolenic acid, esters of linoleic acid, and combinations thereof. The salts and alkaline salts here in refer to those regularly used organic or inorganic salts that are acceptable for pharmaceutical use. Non-limiting exemplary linolenic acids include gamma-linoleic acid and dihomo-gamma-linolenic acid.

The fatty acids of the present inventive subject matter may be from any source, including, without limitation, natural or synthetic oils, fats, waxes or combinations thereof. Moreover, the fatty acids herein may be derived, without limitation, from non-hydrogenated oils, partially hydrogenated oils, fully hydrogenated oils or combinations thereof. Non-limiting exemplary sources of fatty acids include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, shea butter or combinations thereof. Specific non-limiting exemplary fish or marine oil sources include shellfish oil, tuna oil, mackerel oil, salmon oil, menhaden, anchovy, herring, trout, sardines, oils derived from seaweed or kelp, or combinations thereof. Preferably, the source of the fatty acids is fish or marine oil, soybean oil or flaxseed oil.

Linolenic acid is an important precursor of the omega-3 family of fatty acids. The body requires this fatty acid to make eicosapentaenoic acid (EPA) and docosahexaenoic acid (DHA). Many body tissues require EPA and DHA. DHA is especially important in the retina and in the cerebral cortex of the brain. Half of the DHA in a fetus's body accumulates in the brain before birth, and half after birth, an indication of the importance of fatty acids to the fetus during pregnancy and then to the young infant during lactation.

The fatty acids in the present compositions are derived from both plant and animal sources. Combinations of both plant and marine sources of fatty acids are beneficial, because plant derived sources contain only the omega-3 and omega-6 precursors linolenic and linoleic acids, while marine sources contain EPA and DHA. Thus, while the body transforms the plant derived precursors for use, it utilizes the immediately available marine sources of EPA and DHA.

The compositions of the present inventive subject matter may include a vitamin E compound or derivative thereof. The derivatives of vitamin E include, without limitation, alpha-tocopherol, tocopherol, tocotrienol, as well as compounds formed from vitamin E which are structurally distinct from vitamin E, but which retain the active function of vitamin E. Non-limiting examples of such derivatives include: salts of vitamin E, alkaline salts of vitamin E, chelates of vitamin E, combinations thereof and the like. The vitamin E may be present in a single form or in various different forms in combination within the present compositions.

The compositions of the present inventive subject matter may optionally include one or more of the following vitamins or derivatives thereof, without limitation: vitamin B, thiamin, thiamin pyrophosphate, vitamin B₂, riboflavin, flavin mononucleoride, flavin adenine dinucleotide, vitamin B₃, niacin, nicotinic acid, nicotinamide, niacinamide, nicotinamide adenine dinucleotide, tryptophan, biotin, pantothenic acid, vitamin B₁₂, cobalamin, methylcobalamin, deoxyadenosylcobalamin, vitamin C, ascorbic acid, vitamin A, retinol, retinal, retinoic acid, beta-carotene, vitamin D, calciferol, cholecalciferol, dihydroxy vitamin D, 1,25-dihydroxycholecalciferol, 7-dehyrdocholesterol, vitamin K, menadione, menaquinone, phylloquinone, and naphthoquinone.

The compositions of the present inventive subject matter may optionally include one or more of the following minerals and/or trace minerals or derivatives thereof, without limitation: phosphornus, potassium, sulfur, sodium, docusate sodium, chloride, magnesium, magnesium stearate, magnesium carbonate, magnesium oxide, magnesium hydroxide, magnesium sulfate, manganese, copper, iodide, zinc, chromium, molybdenum, fluoride, selenium, molybdenum, cobalt and combinations thereof and derivatives thereof, without limitation. Non-limiting exemplary derivatives of mineral compounds include salts, alkaline salts, esters and chelates of any mineral compound.

The compositions of the present inventive subject matter may optionally include one or more of the following drug categories, in nonteratogenic formulation, without limitation: analgesics, such as acetaminophen, antacids, calcium antacids, magnesium antacids, antibiotics, antihistamines, salicylates, hormonal agents and the like.

The present inventive subject matter may include an edible oil such as one of the following non-limiting examples: seed oil, nut oil, fish oil, vegetable oil, safflower oil, sunflower oil, olive oil, soybean oil, corn oil, safflower oil, olive oil, soybean oil, corn oil, peanut oil, cotton seed oil, palm oil, cocoa oil, coconut oil, flax seed oil, palm kernel oil, canola oil, grape seed oil, walnut oil, sesame oil, cod liver oil, tuna oil, salmon oil, mackerel oil, oils derived from seaweed and kelp, and combinations thereof and derivatives thereof.

The present inventive subject matter may include a polymer, such as one of the following non-limiting examples: polyethylene glycol, propylene glycol, glycerin, polyvinylpyrrolidone, lecithin, PEO, polymeric cellulose esters, copolymeric cellulose esters, cellulose derivatives, acrylate, hydrogenated vegetable oils, natural and synthetic waxes and combinations thereof.

The present inventive subject matter may further include a surfactant such as sodium lauryl sulfate, synthetic ionic surfactant, a synthetic nonionic surfactant, a nonsynthetic ionic surfactant, a nonsynthetic nonionic surfactant, polysorbate 80, polysulfated glucosoglycans, glucosaminoglycans, mucopolysaccharides, derivatives and mixtures thereof and the like, without limitation.

It is also possible in the nutritional composition of the present inventive subject matter for the dosage form to combine various forms of release, which include, without limitation, immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is performed using well known procedures and techniques available to the ordinary artisan. Each of these specific techniques or procedures does not constitute an inventive aspect of this inventive subject matter.

The methods of the present inventive subject matter contemplate dosage forms involving the administration of a nutritional composition in a single dose during a 24 hour period of time, a double dose during a 24 hour period of time, or more than a double dose during a 24 hour period of time. The dosing may be taken simultaneously or at different times depending on the prescribed dosage.

The present inventive subject matter contemplates the use of pharmaceutically acceptable carriers that may be prepared from a wide range of materials. Without being limited thereto, such materials include diluents, binders and adhesives, lubricants, plasticizers, disintegrants, colorants, bulking substances, flavorings, sweeteners, fragrances, aromatics, edible oils, polymers and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives such as whey, starches, and derivatives, as well as other conventional binders well known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, cronotic acid, propylene glycol, butyl phthalate, dibutyl sebacate, caster oil and mixtures thereof, without limitation. As is evident, the plasticizers may be hydrophobic as well as hydrophilic in nature. Water-insoluable hydrophobic substances, such as diethyl phthalate, diethyl sebacate and caster oil are used to delay the release of water-soluble vitamins, such as vitamin B₆ and vitamin C. In contrast, hydrophilic plasticizers are used when water-insoluble vitamins are employed which aid in dissolving the encapsulated film, making channels in the surface, which aid in nutritional composition release.

Flavorings utilized in the nutritional supplements of the present inventive subject matter can be in the form of flavored extracts, volatile oils, and any other commercially available flavoring, without limitation. Nonlimiting examples of flavorings include: pure anise extract, pure vanilla extract, pure lemon extract, pure orange extract, pure peppermint extract, pure spearmint extract, pure ginger extract, imitation banana extract, imitation cherry extract, imitation strawberry extract, imitation raspberry extract, imitation pineapple extract, imitation peach extract, imitation apple extract, imitation coconut extract, vanillin, imitation guava extract, imitation mango extract, balm oil, bay oil, bergamot oil, cinnamon oil, cherry oil, clove oil, peppermint oil, spearmint oil, cedarwood oil, cocoa oil derivatives thereof and combinations thereof.

The compositions of the present inventive subject matter contemplate formulations of various viscosities. The viscous stresses in liquids arise from intermolecular reaction. The concept of viscosity in relation to soft gelatin medicament formulations is important when it is considered that viscosity is used as an index of the suitability of a particular formulation for a particular purpose, i.e., the suitability of a biologically-active core for insertion into a soft gelatin shell.

The centipoise unit is frequently used to measure the dynamic viscosity of mobile liquids and is the unit basis contemplated by the present inventive subject matter. The formal definition of viscosity is derived from a Newtonian theory, wherein under conditions of parallel flow, the shearing stress is proportional to the velocity gradient. If the force acting on each of the two planes of area A parallel each other, moving parallel to each other with a relative velocity V, and separated by a perpendicular distance X, be denoted by F, the shearing stress is F/A and the velocity gradient, which will be linear for a true liquid, is V/X. Thus, F/A=ηV/X, where the constant η is the viscosity coefficient or dynamic viscosity of the liquid. Van Nostrand's Scientific Encyclopedia, 2891 (6^{th} Ed. 1983).

Formulations falling within the scope of the present inventive subject matter may be prepared by methods well known to those of skill in the art, without limitation. For example, without limitation, formulations falling within the scope of the present inventive subject matter may be prepared by dispersing the active substance in an appropriate vehicle, such as vegetable oil or the like, to form a high viscosity mixture. In one embodiment of the present invention the inventive subject matter is prepared by dispersing the active substance in a vehicle including a saturated oil, for example mineral oil. Preferably, the viscosity of the mixture would range from about 1,000 centipoise to about 1.5 million centipoise. Even more preferably, the viscosity of the mixture would range from about 20,000 centipoise to about 130,000 centipoise. Preferably, the viscosity of the mixture would range from about 20,000 centipoise to about 60,000 centipoise. This mixture is then encapsulated with a gelatin based film using technology and machinery known to persons of ordinary skill in the art. The industrial units so formed are then dried to a constant weight and stored for future use.

In a preferred embodiment of the present invention the soft gel shell is formed from at least about 175 bloom gelatin. 175 bloom gelatin provides improved viscosity during the encapsulation process, allowing for more consistent injection wedge temperatures. This ultimately results in improved seals and reduced leakage.

In yet another alternative embodiment, the compositions of the present invention may be utilized in combination with at least one herbal based supplement, as are well known in the art.

The forgoing is considered as illustrative only of the principles of the inventive subject matter. Further, since numerous modification and changes will readily occur to those skilled in the art, it is not desired to limit the inventive subject matter to the exact construction and operation shown and described, and accordingly all suitable modifications and equivalents may be restored to, falling within the scope of the inventive subject matter.

The following examples are illustrative of preferred embodiments of the inventive subject matter and are not to be construed as limiting the inventive subject matter thereto. All percentage are based on the percent by weight of the final delivery system or formulation prepared unless otherwise indicated and all totals equal 100% by weight.

### EXAMPLES

Preparation of Soft Gel Nutritional Supplement

### Example 1

The following compositions were used to prepare soft gelatin prenatal supplements:

Calcium Carbonate 150 mg

Omega-3 Essential Fatty Acid from fish oil 150 mg

Carbonyl Iron 27 mg

Linolenic acid 30 mg

Linoleic acid 30 mg

Sunflower oil 30 mg

Vitamin C 25 mg

Vitamin B6 25 mg

Folic acid 1 mg

Vitamin D3 170 IU

Vitamin E 30 IU

A soft gelatin supplement was prepared by first combining mineral oil and soybean oil in a first vessel and blending it to form a uniform oil mixture, heating the oil mixture to 45 degrees Celsius, and then adding propylene glycol. In a second vessel preheated to 70 degrees Celsius, yellow beeswax and soybean oil were added and blended until a uniform wax mixture was formed. The wax mixture was cooled to 35 degrees Celsius and then added to the oil mixture. To this combined oil and wax mixture the active ingredients listed above were then added and blended together to form a uniform biologically active mixture. The mixture was then cooled to 30 degrees Celsius to form a viscous biologically active core composition, after which time the composition was ready for encapsulation in a soft gelatin shell.

A soft gelatin shell was prepared by heating purified water in a suitable vessel and then adding 175 bloom gelatin. This water gelatin mixture was mixed until the gelatin was fully dissolved, and then glycerin, preservative, one or more flavors, and one or more colorants were added. This gelatin mixture was blended well and cooled. The shells were then filled with the core composition and formed in accordance with soft gelatin techniques commonly used and well known to persons of skill in the art. The resulting soft gelatins were recovered and stored for future use.

The softgels of Examples 2 and 3 were formed by the same method as described for Example 1.

### Example 2

Calcium (from tribasic calcium phosphate 34% Ca) 150 mg

Omega-3 Essential Fatty Acid (from fish oil, 20% EPA/48% DHA) 300 mg

Iron (as carbonyl iron 98% Fe) 27 mg

Linolenic acid (from flaxseed oil NLT 45% linolenic) 30 mg

Linoleic acid (from flaxseed oil NLT 17% linoleic & sunflower oil NLT 65% linoleic) 30 mg

Vitamin C (from ester-C 80% Vit. C) 25 mg

Vitamin B6 (as pyridoxine HCl) 25 mg

Folic acid 1 mg

Vitamin D3 (from cholecalciferol 1 mm IU/g) 170 IU

Vitamin E (from tocopheryl acetate 980 IU/g) 30 IU

### Example 3

Calcium (from tribasic calcium phosphate) 150 mg

Omega-3 Essential Fatty Acid from fish oil 150 mg

Carbonyl Iron 27 mg

Linolenic acid 30 mg

Linoleic acid 30 mg

Sunflower oil 30 mg

Vitamin C 25 mg

Vitamin B6 25 mg

Folic acid 1 mg

Vitamin D3 170 IU

Vitamin E 30 IU

The invention being thus described, it will be apparent that the same may be varied in many ways. Such variations are not to be regarded as a departure from the spirit and scope of the invention, and all such modifications are intended to be within the scope of the appended claims.

1. A nutritional supplement comprising:
at least one essential fatty acid selected from the group consisting of essential fatty acids, precursors thereof, derivatives thereof and mixtures thereof; and
at least one pharmaceutically acceptable iron compound;
wherein said nutritional supplement is provided in a capsule.

2. The nutritional supplement of claim 1 wherein the capsule is a soft gelatin shell dosage form.

3. The nutritional supplement of claim 1 wherein the essential fatty acid includes an essential fatty acid selected from the group consisting of omega-3 fatty acids, omega-6 fatty acids and mixtures thereof.

4. The nutritional supplement of claim 1 wherein said pharmaceutically acceptable iron compound is incorporated into said nutritional supplement.

5. The nutritional supplement of claim 1 further including at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.

6. A nutritional supplement comprising:
at least one essential fatty acid selected from the group consisting of essential fatty acids, precursors thereof, derivatives thereof and mixtures thereof; and
at least one pharmaceutically acceptable iron compound that is non-reactive with the at least one essential fatty acid;
wherein said nutritional supplement is provided in a capsule.

7. The nutritional supplement of claim 6 wherein the capsule is a soft gelatin shell dosage.

8. The nutritional supplement of claim 6 wherein the iron compound is carbonyl iron.

9. The nutritional supplement of claim 6 wherein the iron compound is an encapsulated iron compound.

10. The nutritional supplement of claim 6 further including at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.

11. The nutritional supplement of claim 6 wherein the essential fatty acid includes an essential fatty acid selected from the group consisting of omega-3 fatty acids, omega-6 fatty acids and mixtures thereof.

12. The nutritional supplement of claim 6 wherein the essential fatty acid includes an omega-3 fatty acid.

13. The nutritional supplement of claim 12 wherein the essential fatty acid is an omega-3 fatty acid.

14. The nutritional supplement of claim 6 wherein the essential fatty acid comprises; about 10 mg to 1000 mg of a first fatty acid compound for each compound selected from the group consisting of a linoleic acid compound, a linolenic acid compound, a derivative thereof and a combination thereof; about 10 mg to 1000 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:0.01 to 10.

15. The nutritional supplement of claim 6 wherein the essential fatty acid comprises; about 50 mg to 500 mg of a first fatty acid compound for each compound selected from the group consisting of a linoleic acid compound, a linolenic acid compound, a derivative thereof and a combination thereof; about 100 mg to 500 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:0.09 to 2.5.

16. The nutritional supplement of claim 6 wherein the essential fatty acid comprises; about 100 mg to 300 mg of a first fatty acid compound for each compound selected from the group consisting of a linoleic acid compound, a linolenic acid compound, a derivative thereof and a combination thereof; about 100 mg to 300 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:1 to 2.

17. The nutritional supplement of claim 6 wherein the at least one essential fatty acid includes at least one fatty acid selected from the group consisting of linolenic acid, linoleic acid, precursors thereof, derivatives thereof and mixtures thereof.

18. The nutritional supplement of claim 6 wherein the at least one essential fatty acid includes at least one essential fatty acid selected from the group consisting of arachidonic acid, docosahexanoic acid, eicosapentanoic acid, precursors thereof, derivatives thereof and mixtures thereof.

19. The nutritional supplement of claim 6 further including folic acid, folate, a derivative or metabolite thereof.

20. The nutritional supplement of claim 19 wherein the folic acid, folate, a derivative or metabolite thereof is less than about 5 mg.

21. The nutritional supplement of claim 19 wherein the folic acid, folate, a derivative or metabolite thereof is from about 0.4 mg to about 5 mg.

22. The nutritional supplement of claim 6 further including calcium.

23. The nutritional supplement of claim 22 wherein the calcium is selected from the group consisting of calcium carbonate, dicalcium phosphate, tricalcium phosphate and mixtures thereof.

24. The nutritional supplement of claim 6 further comprising vitamin C.

25. The nutritional supplement of claim 24, wherein said vitamin C is comprised of calcium ascorbate and calcium threonate.

26. The nutritional supplement of claim 6 further comprising vitamin B6.

27. The nutritional supplement of claim 6 wherein the at least one essential fatty acid and the at least one pharmaceutically acceptable iron compound are dissolved in a vehicle including a saturated oil.

28. A nutritional supplement comprising:
about 150 mg of tribasic calcium phosphate;
about 300 mg of omega-3 essential fatty acid from fish oil;
about 27 mg of carbonyl iron;
about 30 mg of linolenic acid;
about 30 mg of linoleic acid;
about 30 mg of sunflower oil;
about 25 mg of vitamin C;
about 25 mg of vitamin B6;
about 1 mg of folic acid, folate, a derivative or metabolite thereof;
about 170 IU of vitamin D3; and
about 30 IU of vitamin E.

29. The nutritional supplement of claim 28 wherein the capsule is a soft gelatin shell dosage form.

30. A method of making a nutritional supplement for administration to said pregnant or lactating woman, the method comprising:
dispersing at least one essential fatty acid selected from the group consisting of essential fatty acids, precursors thereof, derivatives thereof and mixtures thereof with;
at least one pharmaceutically acceptable iron compound into a vehicle to form a viscous mixture; and
encapsulating the viscous mixture into a capsule.

31. The nutritional supplement of claim 30 wherein the capsule is a soft gelatin shell dosage form.

32. The method of claim 30 wherein the essential fatty acid includes an essential fatty acid selected from the group consisting of omega-3 fatty acids, omega-6 fatty acids and mixtures thereof.

33. The method of claim 30 wherein the at least one essential fatty acid and the at least one pharmaceutically acceptable iron compound are dissolved in a vehicle including a saturated oil.

34. The method of claim 30 wherein the nutritional supplement further includes at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.

35. A method of making a nutritional supplement for administration to said pregnant or lactating woman, the method comprising:
ispersing at least one essential fatty acid selected from the group consisting of essential fatty acids, precursors thereof, derivatives thereof and mixtures thereof with at least one pharmaceutically acceptable iron compound that is non-reactive with the at least one essential fatty acid into a vehicle to form a viscous mixture; and
encapsulating the viscous mixture into a soft gelatin shell dosage.

36. The method of claim 35 wherein the capsule is a soft gelatin shell dosage form.

37. The method of claim 35 wherein the iron compound is carbonyl iron.

38. The method of claim 35 wherein the iron compound is an encapsulated iron compound.

39. The nutritional supplement of claim 35 further including at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.

40. The method of claim 35 wherein the essential fatty acid includes an essential fatty acid selected from the group consisting of omega-3 fatty acids, omega-6 fatty acids and mixtures thereof.

41. The method of claim 35 wherein the essential fatty acid includes an omega-3 fatty acid.

42. The method of claim 35 wherein the essential fatty acid is an omega-3 fatty acid.

43. The method of claim 35 wherein the essential fatty acid comprises; about 10 mg to 1000 mg of a first fatty acid compound for each compound selected from the group consisting of a linoleic acid compound, a linolenic acid compound, a derivative thereof and a combination thereof; about 10 mg to 1000 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:0.01 to 10.

44. The method of claim 35 wherein the essential fatty acid comprises; about 50 mg to 500 mg of a first fatty acid compound for each compound selected from the group consisting of a linoleic acid compound, a linolenic acid compound, a derivative thereof and a combination thereof; about 50 mg to 500 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:0.09 to 2.5.

45. The method of claim 35 wherein the essential fatty acid comprises; about 100 mg to 300 mg of a first fatty acid compound for each compound selected from the group consisting of a linoleic acid compound, a linolenic acid compound, a derivative thereof and a combination thereof; about 100 mg to 300 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:1 to 2.

46. The method of claim 35 wherein the at least one essential fatty acid includes at least one fatty acid selected from the group consisting of linolenic acid, linoleic acid, precursors thereof, derivatives thereof and mixtures thereof.

47. The method of claim 35 wherein the at least one essential fatty acid includes at least one essential fatty acid selected from the group consisting of arachidonic acid, docosahexanoic acid, eicosapentanoic acid, precursors thereof, derivatives thereof and mixtures thereof.

48. The method of claim 35 further including folic acid, folate, a derivative or metabolite thereof.

49. The method of claim 48 wherein the folic acid, folate, a derivative or metabolite thereof is less than about 5 mg.

50. The method of claim 48 wherein the folic acid, folate, a derivative or metabolite thereof is from about 0.4 mg to about 5 g.

51. The method of claim 35 further including calcium.

52. The method of claim 51 wherein the calcium is selected from the group consisting of dicalcium phosphate and tricalcium phosphate.

53. The method of claim 35, wherein the nutritional supplement further comprises vitamin C.

54. The method of claim 53, wherein the vitamin C is selected from the group consisting of calcium ascorbate, calcium threonate and mixtures thereof.

55. The method of claim 35, wherein the nutritional supplement further comprises vitamin B6.

56. The method of claim 35 wherein the at least one essential fatty acid and the at least one pharmaceutically acceptable iron compound are dissolved in a vehicle including a saturated oil.

57. A method of providing nutritional support to a pregnant or lactating woman and her child, which comprises administering a nutritional supplement for administration to said pregnant or lactating woman, the nutritional supplement comprising:
about 150 mg of tribasic calcium phosphate;
about 300 mg of omega-3 essential fatty acid from fish oil;
about 27 mg of carbonyl iron;
about 30 mg of linolenic acid;
about 30 mg of linoleic acid;
about 30 mg of sunflower oil;
about 25 mg of vitamin C;
about 25 mg of vitamin B6;
about 1 mg of folic acid, folate, a derivative or metabolite thereof;
about 170 IU of vitamin D3; and
about 30 IU of vitamin E.

58. The nutritional supplement of claim 57 wherein the capsule is a soft gelatin shell dosage form.

59. A method for administering a nutritional supplement, the method comprising:
orally administering to a pregnant or lactating woman a capsule comprising;
at least one essential fatty acid selected from the group consisting of at least one essential fatty acid, at least one essential fatty acid precursor, at least one derivative of an essential fatty acid and mixtures thereof; and
at least one pharmaceutically acceptable iron compound;
wherein said nutritional supplement is provided in a soft gelatin shell dosage.

60. The method of claim 59 wherein the capsule is a soft gelatin shell dosage form.

61. The method of claim 59 wherein the essential fatty acid is selected from the group consisting of omega-3 fatty acids, omega-6 fatty acids and mixtures thereof.

62. The method of claim 59 wherein the at least one essential fatty acid and the at least one pharmaceutically acceptable iron compound are dissolved in a vehicle including a saturated oil.

63. The method of claim 59 wherein the nutritional supplement further includes at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.

64. A method for administering a nutritional supplement, the method comprising:
orally administering to a pregnant or lactating woman a capsule comprising;
at least one essential fatty acid selected from the group consisting of at least one essential fatty acid, at least one essential fatty acid precursor, at least one derivative of an essential fatty acid and mixtures thereof; and
at least one pharmaceutically acceptable iron compound that is non-reactive with the at least one essential fatty acid;
wherein said nutritional supplement is provided in a soft gelatin shell dosage.

65. The method of claim 64 wherein the capsule is a soft gelatin shell dosage form.

66. The method of claim 64 wherein the iron compound is carbonyl iron.

67. The method of claim 64 wherein the iron compound is an encapsulated iron compound.

68. The method of claim 64 wherein the nutritional supplement further includes at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.

69. The method of claim 64 wherein the essential fatty acid is selected from the group consisting of omega-3 fatty acids, omega-6 fatty acids and mixtures thereof.

70. The method of claim 64 wherein the essential fatty acid includes an omega-3 fatty acid.

71. The method of claim 64 wherein the essential fatty acid is an omega-3 fatty acid.

72. The method of claim 64 wherein the essential fatty acid comprises; about 10 mg to 1000 mg of a first fatty acid compound for each compound selected from the group consisting of a linoleic acid compound, a linolenic acid compound, a derivative thereof and a combination thereof; about 10 mg to 1000 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:0.01 to 10.

73. The method of claim 64 wherein the essential fatty acid comprises; about 50 mg to 500 mg of a first fatty acid compound for each compound selected from the group consisting of a linoleic acid compound, a linolenic acid compound, a derivative thereof and a combination thereof; about 50 mg to 500 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:0.09 to 2.5.

74. The method of claim 64 wherein the essential fatty acid comprises; about 100 mg to 300 mg of a first fatty acid compound for each compound selected from the group consisting of a linoleic acid compound, a linolenic acid compound, a derivative thereof and a combination thereof; about 100 mg to 300 mg of a second fatty acid compound selected from the group consisting of a docosahexaenoic acid compound, an omega-3 fatty acid, an omega-2 fatty acid, a derivative thereof and a combination thereof; and wherein the weight ratio of said first fatty acid compound to said second fatty acid compound is about 1:1 to 2.

75. The method of claim 64 wherein the at least one essential fatty acid includes at least one fatty acid selected from the group consisting of linolenic acid, linoleic acid, precursors thereof, derivatives thereof and mixtures thereof.

76. The method of claim 64 wherein the at least one essential fatty acid includes at least one essential fatty acid selected from the group consisting of arachidonic acid, docosahexanoic acid, eicosapentanoic acid, precursors thereof, derivatives thereof and mixtures thereof.

77. The method of claim 64 further including folic acid, folate, a derivative or metabolite thereof.

78. The method of claim 77 wherein the folic acid, folate, a derivative or metabolite thereof is less than about 5 mg.

79. The method of claim 77 wherein the folic acid, folate, a derivative or metabolite thereof is from about 0.4 mg to about 5 mg.

80. The method of claim 64 further including calcium.

81. The method of claim 80 wherein the calcium is selected from the group consisting of calcium carbonate, dicalcium phosphate, tricalcium phosphate and mixtures thereof.

82. The method of claim 64, further comprising vitamin C.

83. The method of claim 82, wherein said vitamin C is comprised of calcium ascorbate and calcium threonate.

84. The method of claim 64, further comprising vitamin B6.

85. The method of claim 64 wherein the at least one essential fatty acid and the at least one pharmaceutically acceptable iron compound are dissolved in a vehicle including a saturated oil.

86. A method for administering a nutritional supplement, the method comprising:
orally administering to a pregnant or lactating woman a capsule comprising;
about 150 mg of tribasic calcium phosphate;
about 300 mg of omega-3 essential fatty acid from fish oil;
about 27 mg of carbonyl iron;
about 30 mg of linolenic acid;
about 30 mg of linoleic acid;
about 30 mg of sunflower oil;
about 25 mg of vitamin C;
about 25 mg of vitamin B6;
about 1 mg of folic acid, folate, a derivative or metabolite thereof;
about 170 IU of vitamin D3; and
about 30 IU of vitamin E.

87. The method of claim 86 wherein the capsule is a soft gelatin shell dosage form.

88. The method of claim 86 wherein the nutritional supplement further includes at least one additive selected from the group consisting of diluents, binders, adhesives, lubricants, plasticizers, disintegrants, colorants, bulking substances, flavorings, sweeteners, fragrances, aromatics, edible oils, polymers, buffers, adsorbents and mixtures thereof.
